(19) United States
(12) Patent Application Publication
(10) Pub. No.: US 2026/0102467 A1
(43) Pub. Date: Apr. 16, 2026

(54) Silk Fibroin/Tea Polyphenol Hydrogel and Preparation Method and Application Thereof

(71) Applicant: The First Affiliated Hospital Of Ningbo University, Ningbo (CN)

(72) Inventors: Kerong Wu, Ningbo (CN); Yaoqi Chen, Ningbo (CN); Xu Cao, Ningbo (CN); Jie Yao, Ningbo (CN); Hua Zhang, Ningbo (CN)

(21) Appl. No.: 19/331,980

(22) Filed: Sep. 17, 2025

(30) Foreign Application Priority Data
Oct. 15, 2024 (WO) PCT/CN2024/125022

(51) Int. Cl. A61K 38/17; A61K 9/06; A61K 36/82

(52) U.S. Cl. CPC A61K 38/1767; A61K 9/06; A61K 36/82

(57) ABSTRACT

A method of preparing silk fibroin/tea polyphenol hydrogel includes steps of preparing silk fibroin by boiling sliced silkworm cocoons in a Na₂CO₃ solution to remove sericin, washing and drying to obtain dry silk, dissolving the dry silk in a LiBr solution, adding glyceryl methacrylate, stirring, filtering the resulting solution and dialyzing, and freeze-drying obtained silk fibroin glycidyl methacrylate solution; preparing silk fibroin hydrogel by irradiating an aqueous solution containing the silk fibroin and a photocrosslinking agent under an ultraviolet light; and preparing the silk fibroin/tea polyphenol hydrogel by immersing the silk fibroin hydrogel in an aqueous solution containing tea polyphenol.

### CROSS REFERENCE OF RELATED APPLICATION

This application is a non-provisional application that claims priority under 35 U.S.C. § 119 to PCT/CN2024/125022, filed date Oct. 15, 2024, wherein the entire content of which is expressly incorporated herein by reference.

### BACKGROUND OF THE PRESENT INVENTION

### Field of Invention

The present invention relates to the technical field of hydrogel preparation, and more particularly to a silk fibroin/tea polyphenol hydrogel and its preparation method and application.

### Description of Related Arts

Hemorrhagic cystitis is a more serious type of cystitis, common in women. The current treatments for hemorrhagic cystitis mainly include the following aspects. One aspect is the antibiotic treatment. For hemorrhagic cystitis caused by bacterial infection, antibiotics are the conventional treatment method. Doctors will choose appropriate antibiotics for treatment based on the results of bacterial culture, which usually has a good effect. The second aspect is the symptomatic treatment. Including hemostatic drugs, analgesics, etc., will help to relieve symptoms and improve the patient's quality of life. The third aspect is the bladder lavage. For severe hemorrhagic cystitis, doctors may perform bladder lavage to clean blood clots and inflammatory substances in the bladder, which helps to relieve symptoms.

However, there are still some problems in treating hemorrhagic cystitis through the above methods. The first problem is the abuse of antibiotics. Long-term and excessive use of antibiotics can easily lead to drug resistance, making some bacteria insensitive to conventional antibiotics and increasing the difficulty of treatment. The second problem is that some patients have repeated attacks. Some patients may have repeated attacks and need long-term regular medication or further exploration of the cause to reduce the recurrence of symptoms. The third problem is that the effect of symptomatic treatment is limited. Although symptomatic treatment can relieve symptoms, it cannot cure the disease. Comprehensive treatment methods are still needed to improve the treatment effect.

Therefore, further research is needed in the future on the etiology and treatment mechanism of hemorrhagic cystitis, and the development of more personalized and effective treatment methods to improve the treatment effect and quality of life of patients.

### SUMMARY OF THE PRESENT INVENTION

An advantage of the present invention is to provides a silk fibroin/tea polyphenol hydrogel and preparation method and application thereof, wherein the silk fibroin/tea polyphenol hydrogel has a good hemostatic effect and is suitable for the treatment of hemorrhagic cystitis.

Another advantage of the present invention is to provide a silk fibroin/tea polyphenol hydrogel and preparation method and application thereof, wherein the silk fibroin/tea polyphenol hydrogel has good adhesion and can closely fit with bladder tissue.

Another advantage of the present invention is to provide a silk fibroin/tea polyphenol hydrogel and preparation method and application thereof, wherein the SFMA/TP hydrogel system can react with urea in urine to change the molecular action in the system, thereby achieving a better hemostatic effect.

Another advantage of the present invention is to provide a silk fibroin/tea polyphenol hydrogel and preparation method and application thereof, wherein the silk fibroin/tea polyphenol hydrogel has good tensile and compressive properties.

Another advantage of the present invention is to provide a silk fibroin/tea polyphenol hydrogel and preparation method and application thereof, wherein the hemorrhagic cystitis treated by the silk fibroin/tea polyphenol hydrogel has no toxic side effects and no recurrence, which is of great significance for the thorough treatment of hemorrhagic cystitis.

According to an aspect of the present invention, the present invention provides a method for preparing a silk fibroin/tea polyphenol hydrogel, comprising the following steps:
(S10) Preparing silk fibroin;
(S20) Preparing silk fibroin hydrogel; and
(S30) Preparing silk fibroin/tea polyphenol hydrogel.

The step (S10) comprises the following steps: (S101) boiling sliced silkworm cocoons in a Na₂CO₃ solution to remove sericin, washing and drying to obtain dry silk; (S102) dissolving the dry silk in a LiBr solution, adding glyceryl methacrylate, stirring, filtering the resulting solution and dialyzing; (S103) freeze-drying obtained silk fibroin glycidyl methacrylate solution and storing the freeze-dried SFMA powder for subsequent use.

In the step (S10), 40 g of sliced cocoons are placed in 1 L of 0.05 M Na₂CO₃ solution and boiled at 100° C. for 30 minutes to remove sericin; then the silk is washed with distilled water for multiple times, and the degummed silk is dried at room temperature. Subsequently, 20 g of the dry silk is dissolved in 100 ml of 9.3 M LiBr solution at 60° C. for 1 hour; 6 mL of glyceryl methacrylate is added to the mixture, and the mixture is stirred at 300 rpm at 60° C. for 3 hours, the resulting solution is filtered with gauze, and dialyzed against distilled water with a 12-14 kDa dialysis tube for 4 days; finally, the silk fibroin glycidyl methacrylate solution is freeze-dried for 48 hours, and the freeze-dried SFMA powder is stored at −80° C. for subsequent use.

In the step (S20), the photocrosslinking agent used includes lithium phenyl-2,4,6-trimethylbenzoylphosphinate, 2-hydroxy-2-methylpropiophenone or 2-hydroxy-4′-(2-hydroxyethoxy)-2-methylpropiophenone.

In the step (S20), the aqueous solution of SFMA and LAP is irradiated under ultraviolet light to prepare SFMA hydrogel.

Wherein in the step (S20), an aqueous solution containing 10% SFMA and 0.2% LAP is irradiated under 405 nm ultraviolet light for 5 minutes to prepare SFMA hydrogel.

In the step (S30), the prepared SFMA hydrogel is immersed in a tea polyphenol aqueous solution for a predetermined time to obtain a silk fibroin/tea polyphenol hydrogel. When used, the silk fibroin and the tea polyphenol are gelled in situ at the lesion site to form the silk fibroin/tea polyphenol hydrogel.

In the step (S30), the prepared SFMA hydrogel is immersed in a 10% TP aqueous solution for 3 hours to obtain a SFMA/TP hydrogel.

The proportion of LAP is in the range of 0.1%-0.5%, and the concentration of tea polyphenols is in the range of 5-20%.

All concentrations of the components described herein are expressed as weight/volume percent (w/v).

According to another aspect of the present invention, the present invention also provides a silk fibroin/tea polyphenol hydrogel, wherein the silk fibroin/tea polyphenol hydrogel is obtained by mixing and reacting the silk fibroin protein hydrogel and tea polyphenol under predetermined conditions.

The silk fibroin/tea polyphenol hydrogel is prepared by the above-mentioned preparation method.

The silk fibroin/tea polyphenol hydrogel is suitable for fitting to the bladder wall.

The silk fibroin/tea polyphenol hydrogel is suitable for treating hemorrhagic cystitis.

The silk fibroin/tea polyphenol hydrogel is prepared by the preparation method according to any one of claims 1 to 8.

The silk fibroin/tea polyphenol hydrogel is suitable for adhering to the mucosal layer of the bladder.

### BRIEF DESCRIPTION OF THE DRAWINGS

FIGS. 1a and 1b are respectively schematic diagrams illustrating the molecular structure and organizational function of silk fibroin and silk fibroin/tea polyphenol hydrogel according to an embodiment of the present invention.

FIGS. 2a and 2b are respectively schematic diagrams of the molecular structure and NMR spectrum of the silk fibroin according to the above embodiment of the present invention.

FIGS. 3a, 3b and 3c are respectively schematic diagrams of the rheological properties of the silk fibroin/tea polyphenol hydrogel according to the above embodiment of the present invention.

FIGS. 4a, 4b and 4c are respectively schematic diagrams of the mechanical properties of the silk fibroin/tea polyphenol hydrogel according to the above embodiment of the present invention.

FIG. 5 is a schematic diagram of a stretching experiment of the silk fibroin/tea polyphenol hydrogel according to the above embodiment of the present invention.

FIGS. 6a and 6b are respectively electron microscope schematic diagrams of the microstructure of the silk fibroin/tea polyphenol hydrogel according to the above embodiment of the present invention.

FIGS. 7a, 7b, 7c, 7d and 7e are respectively schematic diagrams of the adhesion performance test of the silk fibroin/tea polyphenol hydrogel according to the above embodiment of the present invention.

FIGS. 8a, 8b and 8C are respectively schematic diagram of the operation process of intravesical instillation of the silk fibroin/tea polyphenol hydrogel according to the above embodiment of the present invention.

FIG. 9 is a schematic diagram of a dyed section of the silk fibroin/tea polyphenol hydrogel according to the above embodiment of the present invention.

FIG. 10 is a schematic diagram of quantitative analysis of the silk fibroin/tea polyphenol hydrogel in treating mucosal bleeding according to the above embodiment of the present invention.

FIG. 11 is a schematic diagram of the in vitro degradation test results of silk fibroin/tea polyphenol hydrogel (SFMA/TP).

FIG. 12 is a schematic diagram of the HE staining results of silk fibroin/tea polyphenol hydrogel (SFMA/TP) after in vivo degradation.

### DETAILED DESCRIPTION OF THE PREFERRED EMBODIMENT

The following description is used to disclose the present invention so that those skilled in the art can implement the present invention. The preferred embodiments described below are only examples, and those skilled in the art can think of other obvious variations. The basic principles of the present invention defined in the following description can be applied to other embodiments, variations, improvements, equivalents, and other technical solutions that do not deviate from the spirit and scope of the present invention.

### Embodiment 1

A method for preparing the silk fibroin and the silk fibroin/tea polyphenol hydrogel according to a preferred embodiment of the present invention is described as follows.

### (S10) Synthesis of Silk Fibroin (SFMA)

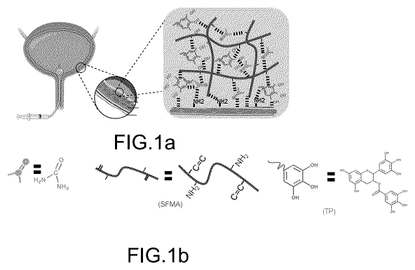

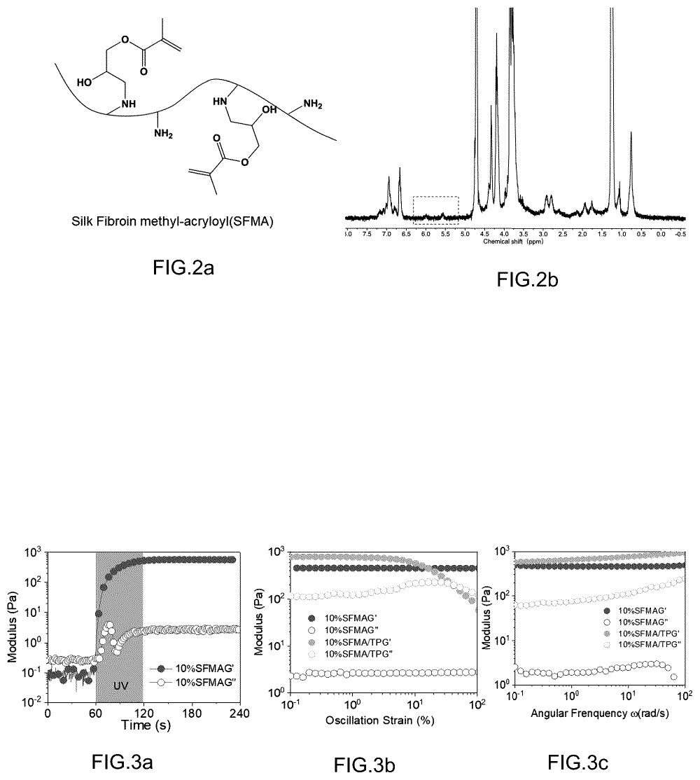

40 g of sliced silkworm cocoons were placed in 1 L of 0.05 M Na₂CO₃ solution and boiled at 100° C. for 30 min to remove sericin, followed by multiple washings with distilled water. The degummed silk was dried at room temperature. Subsequently, 20 g of dry silk was dissolved in 100 mL of 9.3 M LiBr solution at 60° C. for 1 h. Then, 6 mL of glyceryl methacrylate (GMA, 424 mM) was added to the mixture and stirred at 300 rpm at 60° C. for 3 h. The resulting solution was filtered with gauze and dialyzed against distilled water using a 12-14 kDa dialysis tube for 4 days. Finally, the glycidyl methacrylate solution was freeze-dried for 48 h. The freeze-dried SFMA (silk fibroin methacrylate) powder was stored at −80° C. for subsequent use. The obtained SFMA was analyzed by ¹H NMR on a 500 MHz Bruker NMR spectrometer with D₂O as solvent. The molecular structure and organization diagram is shown in FIGS. 1a and 1b, and the ¹H NMR analysis result is shown in FIGS. 2a and 2b. FIG. 2a is a molecular structure diagram of silk fibroin, and FIG. 2b is a nuclear magnetic spectrum of silk fibroin, which proves that the silk fibroin has been successfully synthesized.

### (S20) Synthesis of Silk Fibroin Hydrogel

An aqueous solution containing 10% SFMA and 0.2% LAP was irradiated under 405 nm ultraviolet light for 5 minutes to prepare SFMA hydrogel, wherein LAP is phenyl-2,4,6-trimethylbenzoyl lithium hypophosphite, which is a photocrosslinker for the hydrogel.

### (S30) Synthesis of Silk Fibroin/Tea Polyphenol Hydrogel (SFMA/TP)

The prepared SFMA hydrogel was immersed in a 10% TP aqueous solution for 3 hours and photocured to obtain SFMA/TP hydrogel for in vitro testing. It is worth mentioning that during the application process, the silk fibroin/tea polyphenol hydrogel (SFMA/TP) was formed by in situ gelation at the lesion site. That is, during application, TP was first injected into the bladder, and then the SFMA hydrogel was injected. After photocuring, the silk fibroin/tea polyphenol hydrogel (SFMA/TP) was formed on the surface of the bladder wall to adhere to the mucosal layer of the bladder.

### Embodiment 2

The preparation method of silk fibroin and silk fibroin/tea polyphenol hydrogel is described as follows.

### (S10) Synthesis of Silk Fibroin (SMFA)

40 g of sliced silkworm cocoons were placed in 1 L of 0.05 M Na₂CO₃ solution and boiled at 100 ° C. for 30 min to remove sericin, followed by multiple washings with distilled water. The degummed silk was dried at room temperature. Subsequently, 20 g of dry silk was dissolved in 100 mL of 9.3 M LiBr solution at 60° C. for 1 h. Then, 6 mL of glyceryl methacrylate (GMA, 424 mM) was added to the mixture and stirred at 300 rpm at 60° C. for 3 h. The resulting solution was filtered with gauze and dialyzed against distilled water using a 12-14 kDa dialysis tube for 4 days. Finally, the glycidyl methacrylate solution was freeze-dried for 48 h. The freeze-dried SFMA powder was stored at −80° C. for subsequent use. The obtained SFMA was analyzed by ¹H NMR on a 500 MHz Bruker NMR spectrometer with D₂O as solvent. The molecular structure and organization diagram is shown in FIGS. 1a and 1b, and the ¹H NMR analysis result is shown in FIGS. 2a and 2b. FIG. 2a is a molecular structure diagram of silk fibroin, and FIG. 2b is a nuclear magnetic spectrum of silk fibroin, which proves that the silk fibroin has been successfully synthesized.

### (S20) Synthesis of Silk Fibroin Hydrogel

containing 20% SFMA and 0.5% LAP under 405 nm ultraviolet light for 5 minutes. The LAP is phenyl-2,4,6-trimethylbenzoyl lithium hypophosphite, which is a photocrosslinker for the hydrogel.

### (S30) Synthesis of Silk Fibroin/Tea Polyphenol Hydrogel (SFMA/TP)

The prepared SFMA hydrogel was immersed in a 15% TP (Tea Polyphenol) aqueous solution for 3 hours and photocured to obtain SFMA/TP hydrogel for in vitro testing. It is worth mentioning that during the application process, the silk fibroin/tea polyphenol hydrogel (SFMA/TP) was formed by in situ gelation at the lesion site. That is, during application, TP was first injected into the bladder, and then the SFMA hydrogel was injected. After photocuring, the silk fibroin/tea polyphenol hydrogel (SFMA/TP) was formed on the surface of the bladder wall to adhere to the mucosal layer of the bladder.

### Embodiment 3

The preparation method of silk fibroin and silk fibroin/tea polyphenol hydrogel is described as follows:

(S10) Synthesis of Silk Fibroin (SFMA)

40 g of sliced silkworm cocoons were placed in 1 L of 0.05 M Na₂CO₃ solution and boiled at 100° C. for 30 min to remove sericin, followed by multiple washings with distilled water. The degummed silk was dried at room temperature. Subsequently, 20 g of dry silk was dissolved in 100 mL of 9.3 M LiBr solution at 60° C. for 1 h. Then, 6 mL of glyceryl methacrylate (GMA, 424 mM) was added to the mixture and stirred at 300 rpm at 60° C. for 3 h. The resulting solution was filtered with gauze and dialyzed against distilled water using a 12-14 kDa dialysis tube for 4 days. Finally, the glycidyl methacrylate solution was freeze-dried for 48 h. The freeze-dried SFMA powder was stored at −80° C. for subsequent use. The obtained SFMA was analyzed by ¹H NMR on a 500 MHz Bruker NMR spectrometer with D₂O as solvent. The molecular structure and organization diagram is shown in FIGS. 1a and 1b, and the ¹H NMR analysis result is shown in FIGS. 2a and 2b. FIG. 2a is a molecular structure diagram of silk fibroin, and FIG. 2b is a nuclear magnetic spectrum of silk fibroin, which proves that the silk fibroin has been successfully synthesized.

### (S20) Synthesis of Silk Fibroin Hydrogel

The SFMA hydrogel was prepared by irradiating an aqueous solution containing 10% SFMA and 0.35% LAP under 405 nm ultraviolet light for 4 minutes. The LAP is phenyl-2,4,6-trimethylbenzoyl lithium hypophosphite, which is a photocrosslinker for the hydrogel.

### (S30) Synthesis of Silk Fibroin/Tea Polyphenol Hydrogel (SFMA/TP)

The prepared SFMA hydrogel was immersed in a 20% TP aqueous solution for 3 hours and photocured to obtain SFMA/TP hydrogel for in vitro testing. It is worth mentioning that during the application process, the silk fibroin/tea polyphenol hydrogel (SFMA/TP) was formed by in situ gelation at the lesion site. That is, during application, TP was first injected into the bladder, and then the SFMA hydrogel was injected. After photocuring, the silk fibroin/tea polyphenol hydrogel (SFMA/TP) was formed on the surface of the bladder wall to adhere to the mucosal layer of the bladder.

### Embodiment 4

The preparation method of silk fibroin and silk fibroin/tea polyphenol hydrogel is described as follows:

### (S10) Synthesis of Silk Fibroin (SFMA)

40 g of sliced silkworm cocoons were placed in 1 L of 0.05 M Na₂CO₃ solution and boiled at 100° C. for 30 min to remove sericin, followed by multiple washings with distilled water. The degummed silk was dried at room temperature. Subsequently, 20 g of dry silk was dissolved in 100 mL of 9.3 M LiBr solution at 60° C. for 1 h. Then, 6 mL of glyceryl methacrylate (GMA, 424 mM) was added to the mixture and stirred at 300 rpm at 60° C. for 3 h. The resulting solution was filtered with gauze and dialyzed against distilled water using a 12-14 kDa dialysis tube for 4 days. Finally, the glycidyl methacrylate solution was freeze-dried for 48 h. The freeze-dried SFMA powder was stored at −80° C. for subsequent use. The obtained SFMA was analyzed by ¹H NMR on a 500 MHz Bruker NMR spectrometer with D₂O as solvent. The molecular structure and organization diagram is shown in FIGS. 1a and 1b, and the ¹H NMR analysis result is shown in FIGS. 2a and 2b. FIG. 2a is a molecular structure diagram of silk fibroin, and FIG. 2b is a nuclear magnetic spectrum of silk fibroin, which proves that the silk fibroin has been successfully synthesized.

### (S20) Synthesis of Silk Fibroin Hydrogel

The SFMA hydrogel was prepared by irradiating an aqueous solution containing 10% SFMA and 0.1% LAP under 405 nm ultraviolet light for 6 minutes, wherein LAP is phenyl-2,4,6-trimethylbenzoyl lithium hypophosphite, which is a photocrosslinker for the hydrogel.

### (S30) Synthesis of Silk Fibroin/Tea Polyphenol Hydrogel (SFMA/TP)

The prepared SFMA hydrogel was immersed in a 5% TP aqueous solution for 3 hours and photocured to obtain SFMA/TP hydrogel for in vitro testing. It is worth mentioning that during the application process, the silk fibroin/tea polyphenol hydrogel (SFMA/TP) was formed by in situ gelation at the lesion site. That is, during application, TP was first injected into the bladder, and then the SFMA hydrogel was injected. After photocuring, the silk fibroin/tea polyphenol hydrogel (SFMA/TP) was formed on the surface of the bladder wall to adhere to the mucosal layer of the bladder.

### Embodiment 5

The preparation method of silk fibroin and silk fibroin/tea polyphenol hydrogel is described as follows.

### (S10) Synthesis of Silk Fibroin (SFMA)

40 g of sliced silkworm cocoons were placed in 1 L of 0.05 M Na₂CO₃ solution and boiled at 100° C. for 30 min to remove sericin, followed by multiple washings with distilled water. The degummed silk was dried at room temperature. Subsequently, 20 g of dry silk was dissolved in 100 mL of 9.3 M LiBr solution at 60° C. for 1 h. Then, 6 mL of glyceryl methacrylate (GMA, 424 mM) was added to the mixture and stirred at 300 rpm at 60° C. for 3 h. The resulting solution was filtered with gauze and dialyzed against distilled water using a 12-14 kDa dialysis tube for 4 days. Finally, the glycidyl methacrylate solution was freeze-dried for 48 h. The freeze-dried SFMA powder was stored at −80° C. for subsequent use.

### (S20) Synthesis of Silk Fibroin Hydrogel

The SFMA hydrogel was prepared by irradiating an aqueous solution containing 10% SFMA and 0.2% LAP under 405 nm ultraviolet light for 5 minutes, wherein 2-hydroxy-2-methylpropiophenone was the photocrosslinker of the hydrogel.

### (S30) Synthesis of Silk Fibroin/Tea Polyphenol Hydrogel (SFMA/TP)

The prepared SFMA hydrogel was immersed in a 10% TP aqueous solution for 3 hours and photocured to obtain SFMA/TP hydrogel for in vitro testing. It is worth mentioning that during the application process, the silk fibroin/tea polyphenol hydrogel (SFMA/TP) was formed by in situ gelation at the lesion site. That is, during application, TP was first injected into the bladder, and then the SFMA hydrogel was injected. After photocuring, the silk fibroin/tea polyphenol hydrogel (SFMA/TP) was formed on the surface of the bladder wall to adhere to the mucosal layer of the bladder.

### Embodiment 6

The preparation method of silk fibroin and silk fibroin/tea polyphenol hydrogel is described as follows.

### (S10) Synthesis of Silk Fibroin (SFMA)

40 g of sliced silkworm cocoons were placed in 1 L of 0.05 M Na₂CO₃ solution and boiled at 100° C. for 30 min to remove sericin, followed by multiple washings with distilled water. The degummed silk was dried at room temperature. Subsequently, 20 g of dry silk was dissolved in 100 mL of 9.3 M LiBr solution at 60° C. for 1 h. Then, 6 mL of glyceryl methacrylate (GMA, 424 mM) was added to the mixture and stirred at 300 rpm at 60° C. for 3 h. The resulting solution was filtered with gauze and dialyzed against distilled water using a 12-14 kDa dialysis tube for 4 days. Finally, the glycidyl methacrylate solution was freeze-dried for 48 h. The freeze-dried SFMA powder was stored at −80° C. for subsequent use.

### (S20) Synthesis of Silk Fibroin Hydrogel

The SFMA hydrogel was prepared by irradiating an aqueous solution containing 20% SFMA and 0.5% LAP under 405 nm ultraviolet light for 5 minutes, wherein 2-hydroxy-2-methylpropiophenone was the photocrosslinker of the hydrogel.

### (S30) Synthesis of Silk Fibroin/Tea Polyphenol Hydrogel (SFMA/TP)

The prepared SFMA hydrogel was immersed in a 20% TP aqueous solution for 3 hours and photocured to obtain SFMA/TP hydrogel for in vitro testing. It is worth mentioning that during the application process, the silk fibroin/tea polyphenol hydrogel (SFMA/TP) was formed by in situ gelation at the lesion site. That is, during application, TP was first injected into the bladder, and then the SFMA hydrogel was injected. After photocuring, the silk fibroin/tea polyphenol hydrogel (SFMA/TP) was formed on the surface of the bladder wall to adhere to the mucosal layer of the bladder.

### Embodiment 7

### (S10) Synthesis of Silk Fibroin (SFMA)

40 g of sliced silkworm cocoons were placed in 1 L of 0.05 M Na₂CO₃ solution and boiled at 100° C. for 30 min to remove sericin, followed by multiple washings with distilled water. The degummed silk was dried at room temperature. Subsequently, 20 g of dry silk was dissolved in 100 mL of 9.3 M LiBr solution at 60° C. for 1 h. Then, 6 mL of glyceryl methacrylate (GMA, 424 mM) was added to the mixture and stirred at 300 rpm at 60° C. for 3 h. The resulting solution was filtered with gauze and dialyzed against distilled water using a 12-14 kDa dialysis tube for 4 days. Finally, the glycidyl methacrylate solution was freeze-dried for 48 h. The freeze-dried SFMA powder was stored at −80° C. for subsequent use.

### (S20) Synthesis of Silk Fibroin Hydrogel

The SFMA hydrogel was prepared by irradiating an aqueous solution containing 15% SFMA and 0.4% LAP under 405 nm ultraviolet light for 5 minutes, wherein 2 hydroxy-4′-(2-hydroxyethoxy)-2-methylpropiophenone was a photocrosslinker of the hydrogel.

### (S30) Synthesis of Silk Fibroin/Tea Polyphenol Hydrogel (SFMA/TP)

The prepared SFMA hydrogel was immersed in a 10% TP aqueous solution for 3 hours and photocured to obtain SFMA/TP hydrogel for in vitro testing. It is worth mentioning that during the application process, the silk fibroin/tea polyphenol hydrogel (SFMA/TP) was formed by in situ gelation at the lesion site. That is, during application, TP was first injected into the bladder, and then the SFMA hydrogel was injected. After photocuring, the silk fibroin/tea polyphenol hydrogel (SFMA/TP) was formed on the surface of the bladder wall to adhere to the mucosal layer of the bladder.

### Embodiment 8

### (S10) Synthesis of Silk Fibroin (SFMA)

40 g of sliced silkworm cocoons were placed in 1 L of 0.05 M Na₂CO₃ solution and boiled at 100° C. for 30 min to remove sericin, followed by multiple washings with distilled water. The degummed silk was dried at room temperature. Subsequently, 20 g of dry silk was dissolved in 100 mL of 9.3 M LiBr solution at 60° C. for 1 h. Then, 6 mL of glyceryl methacrylate (GMA, 424 mM) was added to the mixture and stirred at 300 rpm at 60° C. for 3 h. The resulting solution was filtered with gauze and dialyzed against distilled water using a 12-14 kDa dialysis tube for 4 days. Finally, the glycidyl methacrylate solution was freeze-dried for 48 h. The freeze-dried SFMA powder was stored at −80° C. for subsequent use.

### (S20) Synthesis of Silk Fibroin Hydrogel

The SFMA hydrogel was prepared by irradiating an aqueous solution containing 5% SFMA and 0.2% LAP under 405 nm ultraviolet light for 5 minutes, wherein 2 hydroxy-4′-(2-hydroxyethoxy)-2-methylpropiophenone was a photocrosslinker of the hydrogel.

### (S30) Synthesis of Silk Fibroin/Tea Polyphenol Hydrogel (SFMA/TP)

The prepared SFMA hydrogel was immersed in a 15% TP aqueous solution for 3 hours and photocured to obtain SFMA/TP hydrogel for in vitro testing. It is worth mentioning that during the application process, the silk fibroin/tea polyphenol hydrogel (SFMA/TP) was formed by in situ gelation at the lesion site. That is, during application, TP was first injected into the bladder, and then the SFMA hydrogel was injected. After photocuring, the silk fibroin/tea polyphenol hydrogel (SFMA/TP) was formed on the surface of the bladder wall to adhere to the mucosal layer of the bladder.

The silk fibroin/tea polyphenol hydrogel (SFMA/TP) prepared by the above method is suitable for the treatment of hemorrhagic cystitis. As can be seen from FIGS. 1a and 1b, the SFMA/TP hydrogel system can react with urea in urine, causing the molecular action in the system to change.

First, an animal model was established to observe the effect of silk fibroin/tea polyphenol hydrogel (SFMA/TP) in the treatment of hemorrhagic cystitis.

All animal-related procedures were completed in accordance with national and international regulations on animal experiments. The present invention uses SD rats to establish a chemically induced hemorrhagic cystitis model. Cyclophosphamide (CYP) was injected intraperitoneally at a dose of 150 mg/kg, and the model was successfully established 24 hours later. That is, after intraperitoneal injection of rats at a dose of 150 mg/kg, we observed obvious hematuria, bladder bleeding, and submucosal bleeding by naked eye through HE staining.

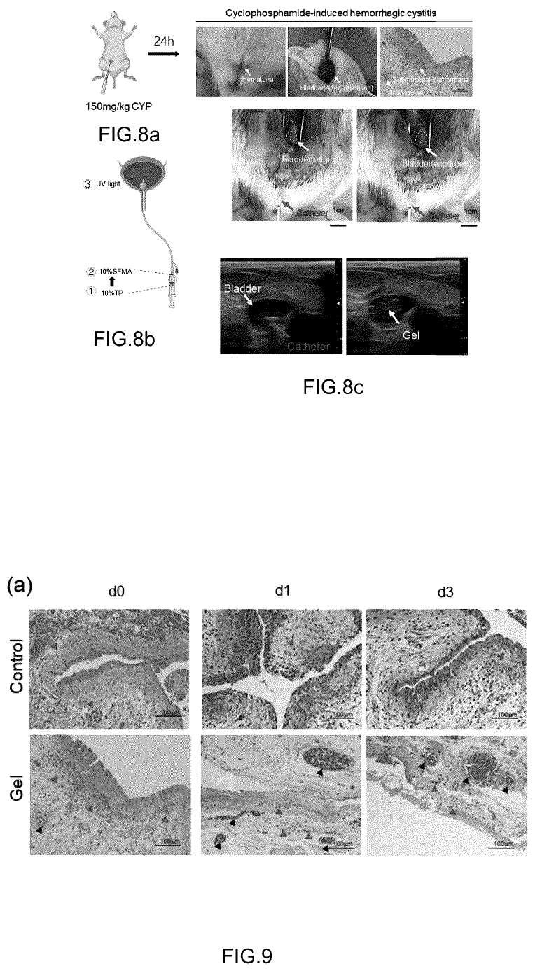

Intravesical injection of SMFA/TP hydrogel for the treatment of hemorrhagic cystitis: After anesthetizing the rat, a midline incision of about 1 cm long was made in the lower abdomen. A urinary catheter was inserted to drain excess urine from the bladder. Subsequently, 0.2 ml of 10% tea polyphenols (TP) was injected, and the bladder was massaged to ensure that the tea polyphenols (TP) were in full contact with the bladder wall. Subsequently, 0.2 ml of 10% SFMA was injected to expand and thin the bladder. The bladder was massaged again and then exposed to UV light to solidify the hydrogel on the bladder wall. Finally, the catheter was removed and the abdominal cavity and skin were sutured. The modeling of hemorrhagic cystitis with silk fibroin/tea polyphenol hydrogel (SFMA/TP) is shown in FIG. 8a.

The rheological properties of silk fibroin/tea polyphenol hydrogel (SFMA/TP) are shown in FIGS. 3a to 3c. As shown in FIGS. 3a to 3c, the storage modulus and loss modulus also increase after the SFMA hydrogel reacts with tea polyphenols (TP), and the silk fibroin/tea polyphenol hydrogel (SFMA/TP) exhibits stronger mechanical properties and adhesion.

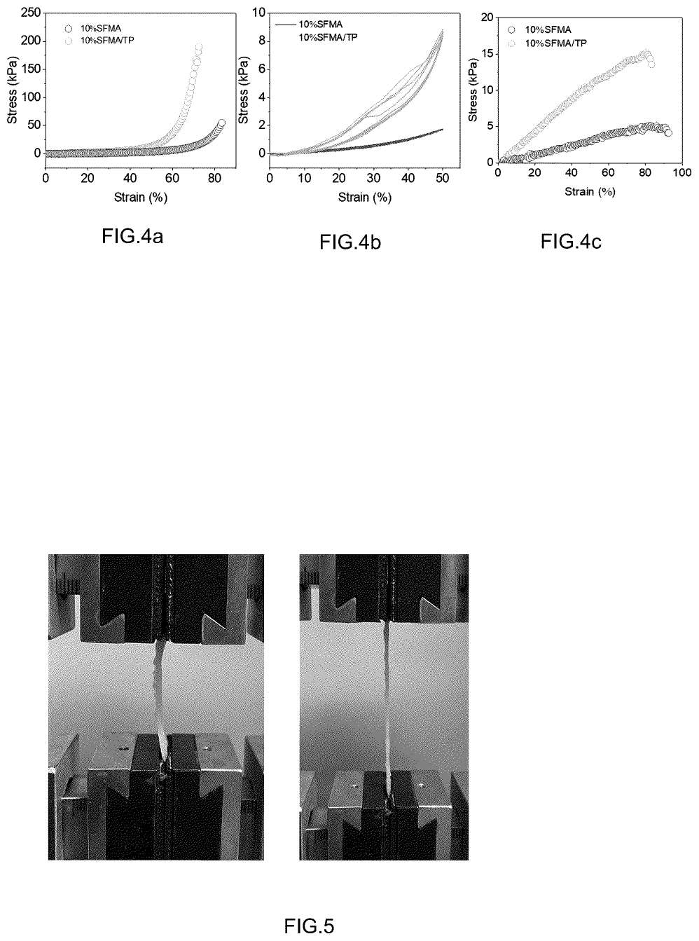

The mechanical properties of silk fibroin/tea polyphenol hydrogel (SFMA/TP) are shown in FIGS. 4a to 4c. As can be seen from FIGS. 4a to 4c, the silk fibroin/tea polyphenol hydrogel (SFMA/TP) generated by the reaction of SFMA hydrogel and tea polyphenol (TP) has significantly improved the compression, cyclic compression and stretching performance of the hydrogel. This enables the silk fibroin/tea polyphenol hydrogel (SFMA/TP) to better adapt to the mechanical effects of bladder contraction and relaxation when used in hemorrhagic cystitis. When the bladder contracts and relaxes, the silk fibroin/tea polyphenol hydrogel (SFMA/TP) can still have good adhesion properties with the bladder tissue.

The tensile test of silk fibroin tea polyphenols hydrogel (SFMA/TP) is shown in FIG. 5. From the test results in FIG. 5, it can be seen that the silk fibroin tea polyphenols hydrogel (SFMA/TP) can be stretched several times and has good tensile properties.

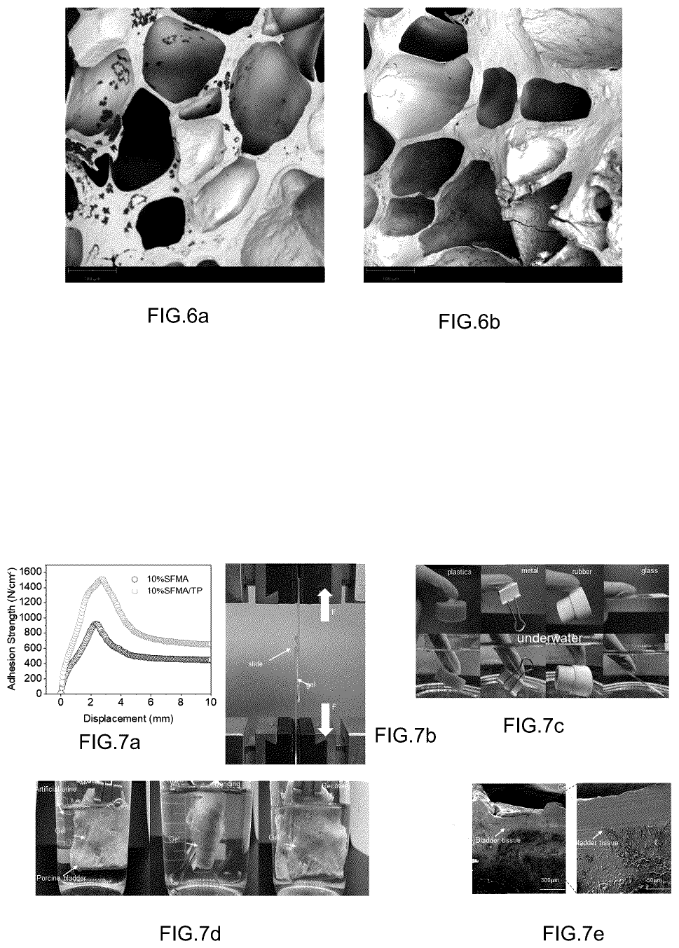

The electron microscope image of silk fibroin/tea polyphenol hydrogel (SFMA/TP) is shown in FIGS. 6a and 6b. It can be seen from FIGS. 6a and 6b that compared with silk fibroin hydrogel (SFMA), silk fibroin/tea polyphenol hydrogel (SFMA/TP) exhibits a more regular and dense microstructure, indicating that silk fibroin/tea polyphenol hydrogel (SFMA/TP) has good mechanical properties such as relaxation and contraction.

The adhesion performance test of silk fibroin tea polyphenols hydrogel (SFMA/TP) and bladder tissue is shown in FIGS. 7a to 7e. As can be seen from FIGS. 7a to 7e, after the silk fibroin tea polyphenols hydrogel (SFMA/TP) interacts with the bladder tissue, the 180-degree glass experiment of the glass slide is shown in FIG. 7a and FIG. 7b, which proves that the adhesion performance of the silk fibroin tea polyphenols hydrogel (SFMA/TP) generated after the reaction of SFMA hydrogel and tea polyphenols is also improved.

In addition, the present invention has tested the adhesion of silk fibroin/tea polyphenol hydrogel (SFMA/TP) to plastics, metals, rubbers, and glass slides, as well as its underwater adhesion ability, the result indicates that silk fibroin/tea polyphenol hydrogel (SFMA/TP) has good adhesion ability.

The present invention further tests the adhesion ability of silk fibroin tea polyphenols hydrogel (SFMA/TP) in artificial urine. As shown in FIG. 7e, it can be seen that silk fibroin tea polyphenols hydrogel (SFMA/TP) can closely fit with the bladder wall and can closely adhere to the bladder when the bladder is deformed.

The hydrogel adhered to the bladder tissue and freeze-dried and then photographed under an electron microscope. The electron microscope image of the adhesion between the hydrogel and the bladder tissue is shown in FIG. 7e. The results show that the hydrogel tissue and the bladder tissue are tightly attached together, confirming that the hydrogel has good adhesion properties.

The operation process and results of hydrogel intravesical instillation are shown in FIGS. 8b and 8c.

Unlike humans, rats have a thinner urethra, and non-invasive intravesical drug delivery through the urethral bladder is difficult. To solve this problem, we used a 3F (diameter=1 mm) catheter for catheterization and intravesical drug delivery, simulating the clinical method of bladder instillation treatment and reducing damage to the bladder. We selected female mice with a shorter and straighter urethra as the animal model, and successfully completed a series of operations such as catheterization and intravesical injection of hydrogel.

After the rats were anesthetized, a midline incision of about 1 cm was made in the lower abdomen. A urinary catheter was inserted to drain excess urine from the bladder. Subsequently, 0.2 ml of 10% TP was injected, and the bladder was massaged to ensure adequate contact between TP and the bladder wall. Subsequently, 0.2 ml of 10% SFMA was injected to expand and thin the bladder. The bladder was massaged again before exposure to UV light. Then, light curing was performed to form SFMA/TP hydrogel on the surface of the bladder wall. Finally, the urinary catheter was removed, and the abdominal cavity and skin were sutured. The filled bladder after perfusion can be observed in FIG. 8c. Ultrasound images show the process of bladder perfusion of hydrogel in real time.

FIG. 9 shows the results of HE staining sections of rats in the silk fibroin/tea polyphenol hydrogel (SFMA/TP) group and the control group at specific time points. As can be seen from FIG. 9, in the silk fibroin/tea polyphenol hydrogel (SFMA/TP) group, the silk fibroin/tea polyphenol hydrogel (SFMA/TP) tissue can be seen adhering to the mucosal layer of the bladder, and a significant reduction in submucosal bleeding can be seen.

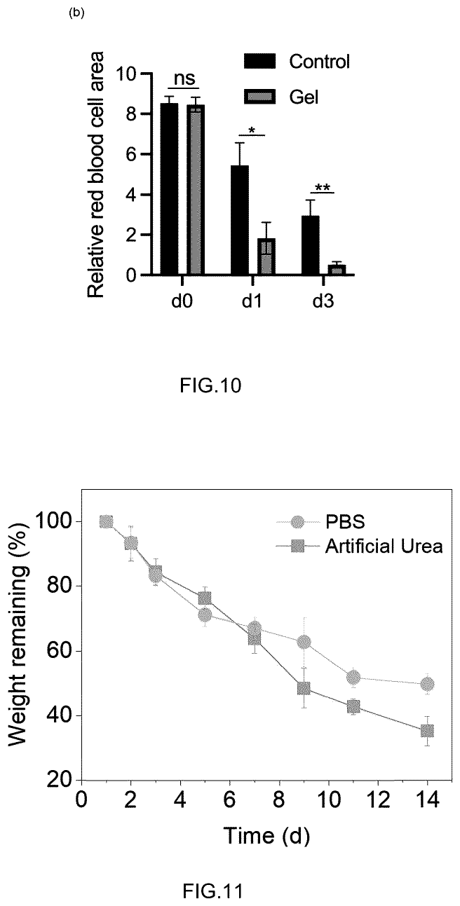

In the treatment of cystitis, the quantitative analysis results of submucosal bleeding in the experimental group and the control group are shown in FIG. 10. FIG. 10 shows the quantitative analysis of submucosal bleeding in the hydrogel group and the control group. The results showed that the silk fibroin/tea polyphenol hydrogel (SFMA/TP) group had obvious hemostatic effect on the 1st and 3rd days after treatment.

Referring to the schematic diagram of the in vitro degradation results of silk fibroin/tea polyphenol hydrogel (SFMA/TP) in FIG. 11, it can be seen from FIG. 11 that the silk fibroin/tea polyphenol hydrogel (SFMA/TP) has good degradation ability in vivo and will not produce toxic side effects in the human body.

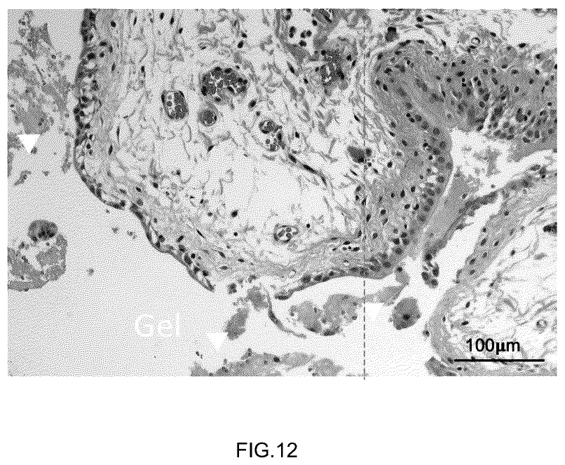

Referring to FIG. 12 which is a schematic diagram of the HE staining results of the silk fibroin/tea polyphenol hydrogel (SFMA/TP) being broken down and discharged into the bladder, wherein the arrow in FIG. 12 indicates the hydrogel, which fully demonstrates the biodegradability and safety of the silk fibroin/tea polyphenol hydrogel (SFMA/TP) in the bladder, which is an important characteristic for ensuring the patency of the urinary tract.

The above results show that silk fibroin/tea polyphenol hydrogel (SFMA/TP) can react with urea in urine and change the molecular action in the system. The silk fibroin/tea polyphenol hydrogel (SFMA/TP) has good adhesion, tensile and compression properties, and can be closely attached when the bladder is deformed. Therefore, it can stop bleeding by adhering silk fibroin/tea polyphenol hydrogel (SFMA/TP) to bladder tissue. It can be found from the experimental group and the control group that silk fibroin/tea polyphenol hydrogel (SFMA/TP) has obvious hemostatic effect in the treatment of hemorrhagic cystitis.

Therefore, the silk fibroin/tea polyphenol hydrogel (SFMA/TP) provided by the present invention can be applied to the treatment of hemorrhagic cystitis.

It should be understood by those skilled in the art that the embodiments of the present invention described above and shown in the accompanying drawings are only examples and do not limit the present invention. The purpose of the present invention has been fully and effectively achieved. The functional and structural principles of the present invention have been demonstrated and explained in the embodiments, and the embodiments of the present invention may be deformed or modified in any way without departing from the principles.

1. A method for preparing a silk fibroin/tea polyphenol hydrogel, comprising the following steps:
(S10) preparing silk fibroin by steps of: (S101) boiling sliced silkworm cocoons in a Na₂CO₃ solution to remove sericin, washing and drying to obtain dry silk; (S102) dissolving the dry silk in a LiBr solution, adding glyceryl methacrylate, stirring, filtering the resulting solution and dialyzing; and (S103) freeze-drying obtained silk fibroin glycidyl methacrylate solution to obtain the silk fibroin which is freeze-dried;
(S20) preparing silk fibroin hydrogel by irradiating an aqueous solution containing the silk fibroin and a photocrosslinking agent under an ultraviolet light; and
(S30) preparing the silk fibroin/tea polyphenol hydrogel by immersing the silk fibroin hydrogel in an aqueous solution containing tea polyphenol.

2. The method according to claim 1, wherein in the step (S10), 40 g of sliced cocoons are placed in 1 L of 0.05 M Na₂CO₃ solution and boiled at 100° C. for 30 minutes to remove sericin; then the silk is washed with distilled water for multiple times, and the degummed silk is dried at room temperature; subsequently, 20 g of the dry silk is dissolved in 100 mL of 9.3 M LiBr solution at 60° C. for 1 hour; 6 mL of glyceryl methacrylate is added to the mixture, and the mixture is stirred at 300 rpm at 60° C. for 3 hours, the resulting solution is filtered with gauze, and dialyzed against distilled water with a 12-14 kDa dialysis tube for 4 days; finally, the obtained silk fibroin glycidyl methacrylate solution is freeze-dried for 48 hours, and the obtained freeze-dried silk fibroin powder is stored at −80° C. for subsequent use.

3. The method according to claim 1, wherein in the step (S20), the photocrosslinking agent comprises lithium phenyl-2,4,6-trimethylbenzoylphosphinate.

4. The method according to claim 2, wherein in the step (S20), the photocrosslinking agent comprises one of lithium phenyl-2,4,6-trimethylbenzoylphosphinate.

5. The method according to claim 1, wherein in the step (S20), by weight-to-volume ration, the aqueous solution containing 5%-20% silk fibroin and 5%-20% photocrosslinking agent.

6. The method according to claim 4, wherein in the step (S20), by weight-to-volume ration, the aqueous solution containing 5%-20% silk fibroin and 0.1%-0.5% photocrosslinking agent.

7. The method according to claim 1, wherein in the step (S20), the aqueous solution containing the silk fibroin and the photocrosslinking agent is irradiated under 405 nm ultraviolet light.

8. The method according to claim 1, wherein in the step (S30), a weight-to-volume ration of the tea polyphenols is in the range of 5%-20%.

9. The method according to claim 2, wherein in the step (S30), a weight-to-volume ration of the tea polyphenols is in the range of 5%-20%.

10. The method according to claim 4, wherein in the step (S30), a weight-to-volume ration of the tea polyphenols is in the range of 5%-20%
